(19) United States
(12) Patent Application Publication
(10) Pub. No.: US 2009/0054686 A1
(43) Pub. Date: Feb. 26, 2009

(54) PROCESS FOR PREPARING CONCENTRATED SOLUTIONS OF SALTS OF ORGANIC ACIDS

(76) Inventors: James Driggers, Canton, GA (US); Stephen F. Driggers, Marietta, GA (US)

Correspondence Address: ATTN: E.J. ASBURY III;TAYLOR BUSCH, LLP, 1600 PARKWOOD CIRCLE, SUITE 200, ATLANTA, GA, 30339, US

(21) Appl. No.: 12/174,612

(22) Filed: Jul. 16, 2008

Related U.S. Application Data
(60) Provisional application No. 60/949,964, filed on Jul. 16, 2007.

(51) Int. Cl. C07C 51/42

(52) U.S. Cl. 562/608; 562/609; 562/606

(57) ABSTRACT

The present invention generally relates to the partial chemical neutralization of organic acids to make compositions used in animal nutrition. More particularly, the present invention relates to the use of a cooled reactor to prepare aqueous compositions of partially neutralized organic acids by a continuous or semi-continuous method of reacting an alkali with an organic acid dispersed or dissolved in an aqueous system. The alkali source is present in an amount less than the stiochometric amount required for complete neutralization of the organic acid.

### CROSS-REFERENCE TO RELATED APPLICATION

This application claims the benefit of U.S. Provisional Application Ser. No. 60/949,964, filed on Jul. 16, 2007, the entirety of which is hereby incorporated herein by reference.

### BACKGROUND OF THE INVENTION

1. Field of the Invention

The present invention generally relates to the partial chemical neutralization of organic acids to make compositions used in animal nutrition. More particularly, the present invention relates to the use of a cooled reactor to prepare aqueous compositions of partially neutralized organic acids by a continuous or semi-continuous method of reacting an alkali with an organic acid dispersed or dissolved in an aqueous system. The alkali source is present in an amount less than the stiochometric amount required for complete neutralization of the organic acid.

2. Description of the Related Art

Organic acids and their salts have usefulness as components of animal feeds and animal feed supplements. Certain organic acids are useful as mold and yeast inhibitors, others are useful in animal nutrition. Organic acids and their salts can be used individually as ingredients or in combination with other substances as components of mixtures of many different substances and additives to give the target animal a nutritionally balanced diet.

Organic acids are used in forms that are un-neutralized, partially neutralized, and completely neutralized. They are used as liquids, powdered salts, or absorbed onto inert carriers. Usually, many tons of animal feed are mixed either continuously or batch wise in relatively short mix times. It is important that ingredients and desired additives for animal feeds be easy and safe to handle, that they exhibit low vapor pressures in order to reduce fumes that may be released during compounding, and that they be of a low degree of corrosiveness.

Though many feeds are compounded using un-neutralized organic acid, the use of salts of these acids on an equivalent strength basis is generally recognized by those skilled in the art to have the following advantages: salts are easier and safer to handle, have lower vapor pressure and are less corrosive than un-neutralized acids. The cations of the particular aqueous partial organic acid salts are ammonium, sodium, and potassium.

Current methods of manufacture of these products involves the charging of a reaction vessel with the appropriate quantity of raw material acid, water and other ingredients and either bubbling an alkaline gas or adding an aqueous solution of alkaline reactant. The reaction of the alkali with the acid is quite violent requiring careful and slow addition of the reactant during all stages of the reaction, especially when the reaction is carried out at elevated temperatures. This reaction is exothermic and generates a considerable amount of heat limiting the rate at which reactants can be safely added. Extreme temperatures above 160° F. contribute to side reactions producing undesirable side by-products and discolored product.

The current processes are limited by the cooling efficiency and capacity of the equipment. Many times the heat generated by the neutralization of the acids is so great that the addition of reactants must be suspended for long periods of time in order to allow the cooling system to remove sufficient heat so that the addition of reactants can be resumed. Many manufacturers require the entire day to manufacture a 5000 gallon batch of finished product. High operating temperature conditions also lead to increased volatile organic acid concentrations in the vapor space over the reacting solution. These organic acid vapors must be contained. Lesser quantities of volatile organic acid will be present in vapor spaces over systems operating at cooler temperatures than those at higher temperatures. The containment of vapors will be simpler at these lower operating temperatures. It is generally recognized that a system operating at a lower temperature poses less of a threat to safety than a system operating at a higher temperature.

It is highly desirable to manufacture as high a concentration on an active acid basis as is stable and practical. Most products of commerce are partially neutralized aqueous solutions of 65% active acid or less. It is desirable to manufactured partially neutralized aqueous solution products of greater concentration, even up to and greater than 90% active acid. This type of product will be more economical to transport on an active acid basis, and less storage space will be required. Higher concentrated products mean less water, or moisture, is added to the feed. Feed ingredients are generally processed and sold as dry, free flowing mixes. Excess moisture is not generally acceptable in a feed mix. More concentrated compositions leave more space by weight in compounded feeds for other desired ingredients to be incorporated.

Accordingly, what is needed in the art is a continuous or semi-continuous process for the manufacture of aqueous partially neutralized salts of organic acids that proceeds at relatively low temperatures, has low capital costs, is repeatable and controllable, flexible, has low operation costs, and produces highly concentrated solutions. The process should be safe for both operating personnel and the environment. It is to such a process of manufacture of aqueous partially neutralized organic acids or aqueous mixture of products containing partially neutralized organic acids that the present invention is directed.

### SUMMARY OF THE INVENTION

The disadvantages of the prior art are overcome by the present invention which, in one aspect, is a process for the production of aqueous partially neutralized salts of organic acids. The process includes a reactor loop, the reactor loop includes a mixing zone and a cooling zone. A circulating solution including an aqueous partially neutralized salt of an organic acid is continuously circulated through the reactor loop. An organic acid and an alkali are added in appropriate proportions within the reactor loop at the mixing zone to the circulating solution to form a reactant mix. The reactant mix then passes to the cooling zone. The organic acid and alkali in the cooled reactant mix then form new aqueous partially neutralized salt of the organic acid.

The circulating solution includes an alkali in an amount less than the stiochometric amount required to form a completely neutralized salt of the organic acid. The organic acid includes; formic, acetic, propionic, citric, maleic, malic, lactic, or any combination thereof. The organic acid includes; fatty acids C₄-C₁₀, fatty acids C₁₂-C₂₂, amino acids, or any combination thereof. The alkali has a cations, the cations of the particular aqueous partial organic acid salts are ammonium, sodium, and potassium.

In another aspect, the process is a continuous process and wherein a portion of the reactant mix is continuously removed from the circulating solution. The process may also be a semi-continuous process and wherein the reactor loop includes a holding tank, the circulating solution passing through the holding tank, and wherein the cooled reactant mix is deposited in the holding tank with the remaining circulating solution. The circulating solution may include an emulsifying agent. The aqueous partially neutralized salts of organic acids include a pH of greater than 3.0, and include greater than 80% by weight organic acid.

In another aspect, the invention is a process for the production of aqueous partially neutralized ammonium salts of organic acids. The process includes a reactor loop having a mixing zone and a cooling zone. A circulating solution including an aqueous partially neutralized ammonium salt of an organic acid is continuously circulated through the reactor loop. An organic acid and an ammonia alkali are added in appropriate proportions within the reactor loop at the mixing zone to the circulating solution to form a reactant mix. The reactant mix then passes to the cooling zone. The organic acid and ammonia alkali in the cooled reactant mix then form new partially neutralized ammonium salt of the organic acid.

In another aspect of the present invention, the organic acid includes; formic, acetic, propionic, citric, maleic, malic, lactic, or any combination thereof. The organic acid may also include; fatty acids C₆-C₁₀, fatty acids C₁₂-C₂₂, amino acids, or any combination thereof.

In another aspect, the ammonium alkali includes anhydrous ammonia. The anhydrous ammonia may be initially in a liquid state and expanded to include a sub-cooled gas prior to addition in the reactor loop. The ammonium alkali may also be aqueous ammonia. The process may be used to manufacture at least one of, ammonium propionate, ammonium acetate, ammonium formate, or combinations thereof.

In another aspect, the circulating solution includes an alkali in an amount less than the stiochometric amount required to form a completely neutralized salt of the organic acid.

In another aspect, the aqueous partially neutralized salts of organic acids include a pH of greater than 3.0, and include greater than 80% by weight organic acid.

In yet another aspect, the circulating solution includes water in excess or the stiochometric amount required to form ammonium hydroxide when the ammonia alkali used is anhydrous ammonia.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE DRAWINGS

FIG. 1 is a process flow diagram for an embodiment of the present invention.

### DETAILED DESCRIPTION OF THE INVENTION

The invention presents a continuous, or semi-continuous, process for the production of aqueous partially neutralized salts of organic acids. The process provides a safe, controllable, repeatable, and flexible means to accomplish an otherwise problematic production operation. The process is rapid, economical, and yields high quality finished products.

In the present invention, safety is achieved by design of equipment and control over operating parameters. Control over operating parameters, control over the rates and the proportion of addition of ingredients, and control over the temperature profile of the reaction as it proceeds, leads to repeatability of product quality. The process is flexible in regards to final pH of the product and the relative proportions of component ingredients. Flexibility in the process is important in that users of these compositions have varying requirements in regards to ingredient compositions and product pH. The process is economical in relation to other methods of manufacture and produces the desired compositions in a rapid manner as order lead times for delivery of these compositions are relatively short in duration.

It is generally recognized that when dealing with an exothermic reaction such as the partial neutralization of an organic acid, it is safer to perform the reaction at a lower temperature than at a higher temperature. As temperature increases, the vapor pressure of organic acids over a solution increases. Reactions performed at higher temperatures must have equipment specially designed to contain the organic acid vapors over the reaction in order to prevent their escape.

It is desirable to manufacture the desired aqueous compositions of partially neutralized organic acids at a controlled temperature. Too high of a temperature of manufacture leads to product discoloration, the possibility of amide formation as a by-product, high vapor pressure of acid over solution in the reaction vessel, and a safety issue. A discolored product, while generally recognized as having little to do with the utility of the product, it does distract from the ascetics of the product and leads to questions from consumers should products vary in color from lot to lot. In fact, many commercial products are dyed in order to mask color variations.

Another reason for controlling temperature is that acid amide formation may be favored by high temperature. For example, acid amides are formed by addition of ammonia to an organic acid with the resulting water removed from the reaction. If the reaction is carried out at or near the boiling point of the solution, locally the reaction conditions may be such that the water is in such low concentration that acid amides may be formed. Acid amides, even in low concentrations, are undesired contaminates in animal feeds and may result in the death of the commercial stock animal.

Aqueous compositions of partially neutralized organic acids are currently manufactured by either: (1) adding organic acids or organic acid solutions to a stirred vessel that contains a solution of the neutralizing alkali, or (2) by adding a solution of the neutralizing alkali to an organic acid or organic acid solution in a stirred tank. This stirred tank may be cooled by means of internal coils, external coils, or a cooling jacket on the sides of the reaction vessel. The problem with these methods is that the temperature of the mixtures during the highly exothermic reaction is difficult to control. Temperatures may fluctuate between various locations in the tank, and the high heat generated by the reaction must be removed using an appropriate cooling method. The extensive cooling system required represents an economic loss in the manufacturing process. Some manufacturers do not apply cooling to the reaction at all, and simply stop the addition of reactants periodically for long periods of time in order to allow the reaction vessel to cool adiabatically. This approach is not continuous, inefficient and does not product uniform product from batch to batch.

The organic acids of interest in the process of the present invention include, but are not limited to the group consisting of those organic acids that are water soluble or easily water dispersible such as formic, acetic, propionic, citric, maleic, malic and lactic; the organic acids that are usually liquid at ordinary temperatures, but usually need an emuslfying or dispersing agent in order to make a uniform mixture with water, such as the group of medium chain fatty acids comprising C₆-C₁₀; organic acids that are solids at ordinary temperatures such as the group of fatty acids comprising C₁₂-C₂₂; and the group comprising certain amino acids.

The organic acids may be reacted singularly, or in mixtures with other organic acids, or with mixtures that include inorganic acids, such as phosphoric acid. The organic acids may also be mixed with ingredients such as nonionic emulsifying agents that may not be chemically affected by the neutralization reaction. For some organic acids, such as the group of medium chain fatty acids comprising C₆-C₁₀, it may be advantageous to incorporate any number of satisfactory emulsifying agents that are known in the art, for example an ethoxylated sorbitan monooleate. For some organic acids, such as Lauric (C₁₂), it is necessary to start the reaction and maintain the reaction at some temperature above the melting point of the organic acid. For these organic acids that must be reacted at some temperature above their melting point, it may be advantageous to incorporate an emulsifying agent.

In the process of the present invention, a range of alkaline reactants may be added to the aqueous organic acids. The cations of the particular aqueous partial organic acid salts are ammonium, sodium, and potassium. In the case of ammonia the reactant will be either anhydrous ammonia or aqueous ammonia, usually available as a commercial aqueous product of 29-31% by weight ammonia. For sodium and potassium, water solutions of 25-50% by weight for sodium hydroxide and 45% by weight potassium hydroxide are commercially available. In all formulations, the alkali source is present in an amount less than the stiochometric amount required for complete neutralization of the organic acid. In all formulations, water is available in the mixture in an amount greater than the stiochometric amount required for complete formation of the salt of the organic acid.

In a first embodiment, the process for manufacturing aqueous compositions of partially neutralized organic acids contains a circulating aqueous solution comprising; (1) water, (2) aqueous alkali, (3) aqueous organic acid, or (4) an aqueous solution of partially neutralized organic acid, or some combination thereof. To this aqueous solution a reactant must be added comprising an aqueous alkali, or an aqueous organic acid.

The circulating aqueous solution flows through a cooled tubular reactor including a mixing zone and a cooling zone. In the mixing zone reactants are introduced into circulating solution to form a reactant mix. The reactants can be introduced into the mixing zone through individual inlet ports, or mixing devices such as mixing “tees”, or mixing “Ys”. The mixing zone is sized to achieve complete and uniform mixing. The method of addition of reactants to the circulating mixture must be done in a manner to ensure un-reacted water is always present at all points in the mixing zone. Stated another way, at all points in the process water is available in the circulating mixture in an amount greater than the stiochometric amount required for complete formation of the salt of organic acid. The presence of un-reacted water is necessary to lessen the possibility of undesired side reactions, such as the formation of amides, or discoloration of the product.

From the mixing zone the resulting reactant mix composition enters the cooling zone which consists of a tubular heat exchanger with a suitable cooling fluid circulating to remove the heat of reaction. The size of the heat exchanger is not critical to the invention, nor is the circulating cooling fluid, as long as both are sufficient to remove the heat of reaction at an acceptable rate. The reactant mix exiting the cooling zone is captured and returned to a product holding tank which is circulated back to the inlet of the mixing zone.

Rates of addition of reactants are controlled by methods known to those skilled in the art utilizing controls such as weigh load cells or flow devices such as meters and controllers that measure and are controlled by parameters such as weight, volume, temperature, or pH. Relative amounts or reactants added is determined by devices such as controllers, that control meters and valves, that are programmed according to formulas for finished compositions which when all individual ingredients are added in the correct proportions will yield the desired product.

The process as described above is semi-continuous in that the volume of aqueous solution within the holding tank is constantly increasing. In an alternative embodiment, the above described process may easily run in a continuous manner. In the case of continuous operation the discharge of the cooling zone would be divided such that a portion equal in weight per unit of time to the total of reactants added into the mixing zone per unit of time be captured and sent outside the system to an external holding tank. The remaining amount exiting the cooling zone would be returned to the holding tank to be circulated through the system again entering the mixing zone. In an alternative embodiment, a discharge from the holding tank may remove a portion equal in weight per unit of time to the total of reactants added into the mixing zone per unit of time. In either approach, the total amount of the mixture in the holding tank and circulating through system in a loop never changes over time. The details for controllers, meters, valves, weight load cells necessary for such a design are known by those familiar in the art.

In both the continuous or semi-continuous embodiment above, the process can be configured to produce a finished product in one continuous process loop. In each embodiment, the key variables in the production of a uniform product remain relatively constant during operation. The total amount of mixture circulating through the system, and rate of reactant addition to the circulating mixture are constant in operation. The temperature of the mixture at any given point in the system is stable, and the heat required to be removed in the cooling zone is a constant quantity. The reactants are diluted in the flow stream of the product loop allowing for simpler incremental adjustment of reactant feeds and reduced temperature excursions. This methodology results in a consistent, repeatable product of high quality.

It is well known to those in the art that the amount heat generated during a aqueous neutralization done by adding reactants together over some period of time is greater at the beginning of the addition than later toward the end of the addition. This is due to the heat of dilution that is generated by the hydrolysis of compounds from more concentrated to dilute states. This heat is in addition to the heat of reaction of the action of the base on the acid. The present invention reduces this heat load somewhat by giving an option of utilizing a circulating solution of some concentration that already contains partially neutralized organic acid salt into which reactants are introduced. This option requires comparatively little dilution, thereby producing less heat of dilution. Then the major heat load is primarily from the heat of reaction.

In order to obtain concentrated solutions of partially neutralized ammonium salts of organic acids with pH greater than 3.0, ie over 80% active acid, it is necessary to utilize anhydrous ammonia as the reactant. In another embodiment of the present invention, the reactant is anhydrous ammonia added to a circulating solution of partially neutralized ammonium salt of organic acid. This embodiment negates partially the additional heat load from the dilution of ammonium hydroxide that would be encountered if anhydrous ammonia was to be added to a stirred vessel containing an aqueous organic acid. In a stirred vessel, anhydrous ammonia chemically reacts first with water to form ammonium hydroxide which generates both a heat of reaction from its formation and additionally a heat of dilution with water. This heat is in addition to the heat of reaction of the acid and ammonia. By circulating a somewhat concentrated solution, the heat of dilution is lessened and the heat load is easier to control. In this embodiment, the process may be used to manufacture ammonium propionate, ammonium acetate, ammonium formate, and combinations of other organic acid ammonium salts.

The use of anhydrous ammonia in a liquid form offers another process advantage. The limiting factor in this reaction is the control of temperature especially at the beginning of the reaction when the reactants are at their maximum concentrations. In operation, prior to addition in the circulating solution, the liquid anhydrous ammonia is allowed to expand into a gas. This is a highly endothermic reaction resulting in sub-cooled ammonia gas. By introducing the sub-cooled ammonia gas as the reactant, much of the heat of reaction is absorbed within the system reducing the amount of external cooling required.

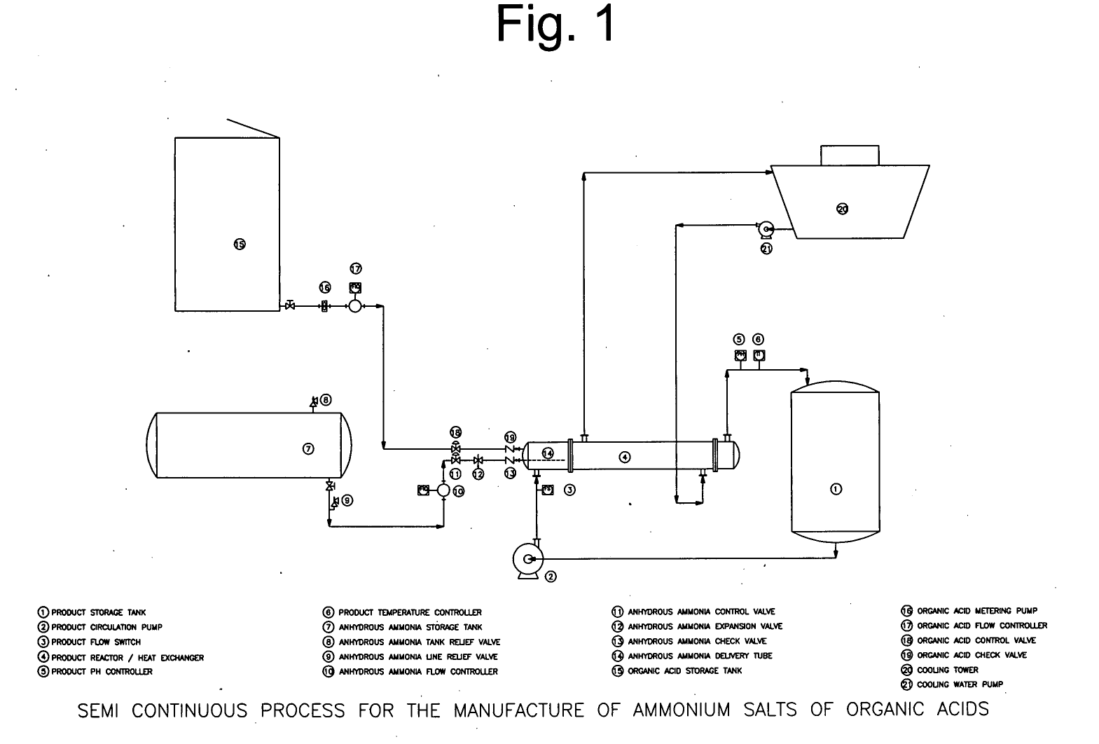

Turning now to the Figures and Tables, FIG. 1 presents a process flow diagram for one embodiment of the present invention. In FIG. 1, the reactant is ammonium in the form of liquid anhydrous ammonium. The process is started by placing enough quantity of finished product in the Product Storage Tank (1) to fill and circulate within the process loop. An appropriate amount of organic acid is placed in the Organic Acid Storage Tank (15). The appropriate quantity of water for the final finished product formulation is added to the Organic Acid Storage Tank (15). The Product Circulation Pump (2) is started, pumping product through the Product Reactor/Heat Exchanger (4) and returning to the Product Storage Tank (1). It is extremely important that the required quantity of water be added to the Organic Acid Storage Tank (15) prior to the reaction by adding it to the acid feed to prevent the formation of Amides due to the dehydration of the product.

Within the process loop there are several control devices for ease of operation and safety: Product Flow Switch (3) is a flow switch interlocked with the Anhydrous Ammonia Control Valve (11) and the Organic Acid Metering Pump (16) and Organic Acid Control Valve (18) to prevent the addition of raw materials in the event of loss of flow within the process flow loop.

To begin the process, cooling water is circulated through the heat exchanger. In this example, the Cooling Water Pump (21) and the Cooling Tower (20) are started. The Organic Acid Control Valve (18) is opened. The Organic Acid Metering Pump (16) is started and the flow rate of acid is set to a predetermined flow rate. The Anhydrous Ammonia Control Valve (11) is opened and the rate of ammonia flow is adjusted with the Anhydrous Ammonia Expansion Valve (12). The ammonia flow rate is adjusted based on the ratio of ammonia to acid in the finished product. As the Liquid Anhydrous Ammonia passes through the expansion valve, the ammonia expands from a liquid to a sub-cooled gas creating a refrigeration effect thus absorbing some of the heat generated during the reaction process.

The process is monitored by the Product PH Controller (5) and the Product Temperature Controller (6). Should the PH increase above the limits, either the acid flow is increased or the flow of the ammonia decreased. Typical PH values for these groups of products is 3.5-5.5. Additionally, temperature is monitored with the Temperature Controller (6). Maximum operating temperature is 140 F. As the temperature increases, the feed rates of ammonia and acid should be reduced. Should the temperature reach the maximum, the ammonia and acid feeds should stop until the loop temperature is stabilized. The finished product is analyzed for various properties such as PH, density, and concentration. Incremental adjustments may be made by adding the appropriate raw material to the process loop.

In an alternative embodiment of the present inventive process, a raw material organic acid-water solution is circulated within the process loop in lieu of a finished product. In this embodiment, the required quantity of raw material acid and water are added to the product tank and circulated within the process loop. Anhydrous ammonia is added to the flow stream in the reactor/heat exchanger in a similar method as above whereas the liquid anhydrous ammonia is fed through an expansion valve allowing the liquid to expand into a sub cooled gas. As in the previous example, the sub-cooled ammonia gas absorbs much of the heat of reaction. The amount of ammonium salt present in the process tank, and the Ph of the circulating mixture increases over time. This alternate process provides many of the same benefits as the previous example except closer process monitoring is required and the process is no longer continuous.

In another alternate embodiment of the present invention, the process of manufacture involves the addition of aqueous ammonia to the process loop in lieu of anhydrous ammonia. This aqueous ammonia solution can be feed in a continuous stream in situ with the acid feed similar to the first embodiment or feed into a circulating stream of acid water solution as in the second alternate embodiment. In this embodiment, the advantage of the additional cooling effect due to the expansion of the liquid anhydrous ammonia into a sub-cooled gas is no longer realized creating additional external cooling requirements.

The preceding embodiments present ammonia as the alkaline reactant. As will be appreciated by those skilled in the art, all embodiments may be used to produce aqueous organic acid solutions using sodium, or potassium. Water solutions of 25-50% by weight of sodium hydroxide and 45% by weight potassium hydroxide are commercially available and may be used as the alkaline reactant in the process.

### EXAMPLE 1

A finished product consisting of 82% by weight formic acid and 10% by weight ammonia with a pH greater than 3.0 was produced by adding in the mixing zone of the present invention, anhydrous ammonia and a formic acid solution in correct proportion, to a circulating aqueous solution of partially neutralized ammonium formate and formic acid. Temperature of the solution exiting the cooling zone was maintained at less than 140° F. or 60° C.

### EXAMPLE 2

A finished product consisting of 91% by weight propionic acid and 4% by weight ammonia with a pH greater than 3.0 was produced by adding in the mixing zone of the present invention, anhydrous ammonia and a propionic acid solution in correct proportion, to a circulating solution of aqueous partially neutralized ammonium propionate and propionic acid. Temperature of the solution exiting the cooling zone was maintained at less than 160° F. or 71° C.

### EXAMPLE 3

A finished product consisting of 68% by weight propionic acid, 2.5% by weight citric acid and 7% by weight ammonia with a pH greater than 4.0 was produced by, adding in the mixing zone of the present invention, anhydrous ammonia in correct proportion, to a circulating aqueous solution that began as an aqueous solution of propionic and citric acids. In this case the correct amount of water, propionic acid, and citric acids were mixed in the holding tank and then circulated through the system as anhydrous ammonia was added until the desired proportion was obtained. Temperature of the solution exiting the cooling zone was maintained at less than 160° F. or 71° C.

### EXAMPLE 4

A finished product consisting of 65% by weight propionic acid with a pH greater than 4.0 was produced by adding aqua ammonia, an aqueous ammonia solution of 29-31% by weight ammonia, to a circulating mixture that began as an aqueous solution of propionic in the mixing zone of the present invention anhydrous ammonia in correct proportion. In this case the correct amount of water, propionic acid, and citric acids were mixed in the holding tank and then circulated through the system as anhydrous ammonia was added until the desired proportion was obtained. Temperature of the solution exiting the cooling zone was maintained at less than 160° F. or 71° C.

All of the manufacturing processes disclosed and claimed herein can be made and executed without undue experimentation in light of the present disclosure. While the compositions and process of this invention have been described in terms of preferred embodiments, it will be apparent to those of skill in the art that variations may be applied to the compositions and process and in the steps, or in the sequence of steps, of the methods described herein without departing from the concept, spirit, and scope of the invention. More specifically, it will be apparent that certain chemical compounds which are both chemically related, and similar physical characteristics, may be substituted for the chemical compounds described herein while the same or similar results would be achieved. All such similar substitutes and modifications apparent to those skilled in the art are deemed to be within the spirit, scope, and concept of the invention.

We claim:

1. A process for the production of aqueous partially neutralized salts of organic acids, the process comprising:
a reactor loop, the reactor loop comprising a mixing zone and a cooling zone;
wherein a circulating solution comprising an aqueous partially neutralized salt of an organic acid is continuously circulated through the reactor loop,
wherein an organic acid and an alkali are added in appropriate proportions within the reactor loop at the mixing zone to the circulating solution to form a reactant mix;
wherein the reactant mix then passes to the cooling zone; and
wherein the organic acid and alkali in the cooled reactant mix then form new aqueous partially neutralized salt of the organic acid.

2. The process of claim 1, wherein the circulating solution comprises an alkali in an amount less than the stiochometric amount required to form a completely neutralized salt of the organic acid.

3. The process of claim 1, wherein the organic acid comprises; formic, acetic, propionic, citric, maleic, malic, lactic, or any combination thereof.

4. The process of claim 1, wherein the organic acid comprises; fatty acids C₄-C₁₀, fatty acids C₁₂-C₂₂, amino acids, or any combination thereof.

5. The process of claim 1, wherein the alkali has a cations, the cations of the particular aqueous partial organic acid salts are ammonium, sodium, and potassium.

6. The process of claim 1, wherein the process is a continuous process and wherein a portion of the reactant mix is continuously removed from the circulating solution.

7. The process of claim 1, wherein the process is a semi-continuous process and wherein the reactor loop comprises a holding tank, the circulating solution passing through the holding tank, and wherein the cooled reactant mix is deposited in the holding tank with the remaining circulating solution.

8. The process of claim 4, wherein the circulating solution comprises an emulsifying agent.

9. The process of claim 1, wherein the aqueous partially neutralized salts of organic acids comprise a pH of greater than 3.0, and comprise greater than 80% by weight organic acid.

10. A process for the production of aqueous partially neutralized ammonium salts of organic acids, the process comprising:
a reactor loop, the reactor loop comprising a mixing zone and a cooling zone;
wherein a circulating solution comprising an aqueous partially neutralized ammonium salt of an organic acid is continuously circulated through the reactor loop,
wherein an organic acid and an ammonia alkali are added in appropriate proportions within the reactor loop at the mixing zone to the circulating solution to form a reactant mix;
wherein the reactant mix then passes to the cooling zone; and
wherein the organic acid and ammonia alkali in the cooled reactant mix then form new partially neutralized ammonium salt of the organic acid.

11. The process of claim 10, wherein the organic acid comprises; formic, acetic, propionic, citric, maleic, malic, lactic, or any combination thereof.

12. The process of claim 10, wherein the organic acid comprises; fatty acids C₆-C₁₀, fatty acids C₁₂-C₂₂, amino acids, or any combination thereof.

13. The process of claim 10, wherein the ammonium alkali comprises anhydrous ammonia.

14. The process of claim 13, wherein the anhydrous ammonia is initially in a liquid state and expanded to comprise a sub-cooled gas prior to addition in the reactor loop.

15. The process of claim 10, wherein the ammonium alkali is aqueous ammonia.

16. The process of claim 10, wherein the process is used to manufacture at least one of; ammonium propionate, ammonium acetate, ammonium formate, or combinations thereof.

17. The process of claim 10, wherein the circulating solution comprises an alkali in an amount less than the stiochometric amount required to form a completely neutralized salt of the organic acid.

18. The process of claim 10, wherein the aqueous partially neutralized salts of organic acids comprise a pH of greater than 3.0, and comprise greater than 80% by weight organic acid.

19. The process of claim 10, wherein the circulating solution comprises water in excess or the stiochometric amount required to form ammonium hydroxide when the ammonia alkali used is anhydrous ammonia.
